SEQUENCE LISTING (1 sequence)

SEQ ID NO: 1 - DNA, 494 bp, Beauveria bassiana
  source 1..494: mol_type = genomic DNA
gagggatcattaccgagttttcaactccctaacccttctgtgaacctacctatcgttgcttcggcggactcgccccagcccggacgcggactggaccagcggcccgccggggacctcaaactcttgtattccagcatcttctgaatacgccgcaaggcaaaacaaatgaatcaaaactttcaacaacggatctcttggctctggcatcgatgaagaacgcagcgaaatgcgataagtaatgtgaattgcagaatccagtgaatcatcgaatctttgaacgcacattgcgcccgccagcattctggcgggcatgcctgttcgagcgtcatttcaaccctcgacctccccttggggaggtcggcgttggggaccggcagcacaccgccggccctgaaatggagtggcggcccgtccgcggcgacctctgcgtagtaatacagctcgcaccggaaccccgacgcggccacgccgtaaaacacccaacttctgaacgt

(19) United States
(12) Patent Application Publication
(10) Pub. No.: US 2025/0179420 A1
(43) Pub. Date: Jun. 5, 2025

(54) Beauveria Bassiana KNU-101 Strain Having Excellent Insecticidal Effect and Sporulation Potential and Use Thereof

(71) Applicant: KYUNGPOOK NATIONAL UNIVERSITY INDUSTRY-ACADEMIC COOPERATION FOUNDATION, Daegu (KR)

(72) Inventors: JAE HO SHIN, Daegu (KR); CHANG EON PARK, Daegu (KR); YEON GYUN JUNG, Daegu (KR); SEUNG DAE CHOI, Gangneung-si (KR); YEONG JUN PARK, Daegu (KR); HYUNG WOO JO, Seongnam-si (KR); MIN JI KIM, Daegu (KR); MIN KYU PARK, Incheon (KR); MIN CHUL KIM, Daegu (KR)

(21) Appl. No.: 19/057,133

(22) Filed: Feb. 19, 2025

Related U.S. Application Data
(62) Division of application No. 17/294,153, filed on Jun. 14, 2021.

(30) Foreign Application Priority Data
Nov. 16, 2018 (KR) 10-2018-0142137
Nov. 14, 2019 (KR) 10-2019-0145696

(51) Int. Cl. C12N 1/14; A01N 63/30; A01P 7/04; C12R 1/645

(52) U.S. Cl. CPC C12N 1/145; A01N 63/30; A01P 7/04; C12R 2001/645

(57) ABSTRACT

The present disclosure relates to a Beauveria bassiana KNU-101 strain. The Beauveria bassiana KNU-101 strain is entomopathogenic, exhibits an excellent insecticidal effect on controlling thrips, which are soil pests that harm crops, and also has excellent sporulation ability. Thus, the Beauveria bassiana KNU-101 strain may be effectively used as an environmentally friendly means for controlling pests.

### CROSS REFERENCE TO RELATED APPLICATIONS

This application is a divisional of application Ser. No. 17/294,153, filed Jun. 14, 2021, which is § 371 national phase application of PCT Application No. PCT/KR2019/015616, filed on Nov. 15, 2019, which claims priority to Korean Application No. 10-2018-0142137 filed on Nov. 16, 2018, and Korean Application No. 10-2019-0145696 filed on Nov. 14, 2019, the entire contents of each of which is incorporated by reference herein.

### STATEMENT REGARDING ELECTRONIC FILING OF A SEQUENCE LISTING

A Sequence Listing in XML format, entitled 1433-48DV_ST26.xml, 2,415 bytes in size, generated on Feb. 18, 2025, and filed herewith, is hereby incorporated by reference into the specification for its disclosures.

### TECHNICAL FIELD

The present disclosure relates to a novel Beauveria bassiana KNU-101 strain (accession number: KCTC 13660BP) having an excellent insecticidal effect and excellent sporulation ability, a method for mass production of the strain, a microbial insecticide formulation containing the strain, and an insecticidal method using the microbial insecticide formulation.

The present disclosure was made with the support of the Korean government under grant number 1395058780 (“The Cooperative Research Program for Agriculture Science and Technology Development”) awarded by Rural Development Administration.

The present disclosure was made with the support of the Korean government under grant number 1545018506 (“The Strategic Initiative for Microbiomes in Agriculture and Food) awarded by Ministry of Agriculture, Food and Rural Affairs.

### BACKGROUND ART

Insects have the largest number of species among currently known organisms on the earth and are known to have excellent adaptability to the environment. Since some of these insects cause a lot of damage to various commercially important agricultural crops, chemical insecticides have been mainly used to control these pests. However, chemical insecticides have disadvantages in that they also kill non-target microorganisms such as beneficial insects and parasites in pests due to the non-specific spectrum of insecticidal ability, and the frequent use of the chemical insecticides promotes the acquisition of resistance of target pests repeatedly exposed to the chemical insecticides, and also chemical insecticide components are harmful to the human body.

In order to reduce the use of chemical insecticides, a method that uses entomopathogenic fungi as an environmentally friendly pest control agent has been used. These entomopathogenic fungi have specific insecticidal activity only against host insects without causing harm to humans and animals, including plants, and thus have the advantage of being able to effectively control target pests.

Meanwhile, Beauveria bassiana is known as a representative entomopathogenic fungus, and it is known that spores thereof dehydrate an insect while destroying the waxy layer of the insect, resulting in death of the insect, and use the corpse of the insect as nourishment to continue the next generation. For this reason, it is known that both oral and contact toxicities of the spores to insects are effective, and the spores may infect various insect hosts. For mass culture of spores of this pest control fungus, solid culture for producing conidia has been mainly used.

However, unlike bacteria, fungi do not produce sufficient amounts of spores despite a long-term culture period of at least 7 days, and hence the commercialization of fungi for controlling pests is being delayed. In addition, Beauveria bassiana strains reported to date are also known to have low sporulation ability that is insufficient for commercial use. Thus, for the commercial use thereof, it is a priority task to select fungi satisfying both the following requirements: having the ability to produce a large amount of spores; and exhibiting excellent insecticidal activity.

Meanwhile, thrips that are typical soil pests lay eggs on soft tissues such as young leaves, stems, and petals, and begin to appear as the temperature rises, damaging the surfaces of stems and leaves. During the flowering period, thrips severely degrade the quality of crops by damaging buds. Crops damaged by thrips show lesions in which the leaves are not straightened and have deformities or the leaf veins constrict. Thrips have a wide range of hosts, including cucumbers, lettuce, tomatoes, eggplants, and chicory, and have a very short generation period from at least 12 days to a maximum of 26 days depending on temperature conditions, and are difficult to control because of their strong fertility.

Although studies on various substances for pest control as described above have been conducted, studies on bio-insecticides still remain insufficient compared to studies on chemical insecticides.

### DISCLOSURE

### Technical Problem

To achieve the above-described object of the present disclosure, one aspect of the present disclosure provides a Beauveria bassiana KNU-101 strain (accession number: KCTC 13660BP) having an insecticidal effect.

According to one embodiment of the present disclosure, the strain may comprise an ITS (Internal Transcribed Spacer Region) nucleotide sequence represented by SEQ ID NO: 1.

Another aspect of the present disclosure provides a method for mass production of a Beauveria bassiana KNU-101 strain (accession number: KCTC 13660BP), the method comprising a step of culturing the strain.

According to one embodiment of the present disclosure, the culturing may be performed at 28° C. to 30° C.

Still another aspect of the present disclosure provides a microbial insecticide formulation containing, as an active ingredient, at least one selected from the group consisting of a Beauveria bassiana KNU-101 strain (accession number: KCTC 13660BP), a culture of the strain, a concentrate of the culture, and a dried product of the culture.

According to one embodiment of the present disclosure, the microbial insecticide formulation may have insecticidal activity against thrips.

Yet another aspect of the present disclosure provides an insecticidal method comprising a step of treating soil or a plant with the microbial insecticide formulation.

Technical Solution

One aspect of the present disclosure provides a Beauveria bassiana KNU-101 strain (accession number: KCTC 13660BP) having an insecticidal effect.

According to one embodiment of the present disclosure, the strain may comprise an ITS (Internal Transcribed Spacer Region) nucleotide sequence represented by SEQ ID NO: 1.

Another aspect of the present disclosure provides a method for mass production of a Beauveria bassiana KNU-101 strain (accession number: KCTC 13660BP), the method comprising a step of culturing the strain.

According to one embodiment of the present disclosure, the culturing may be performed at 28° C. to 30° C.

Still another aspect of the present disclosure provides a microbial insecticide formulation containing, as an active ingredient, at least one selected from the group consisting of a Beauveria bassiana KNU-101 strain (accession number: KCTC 13660BP), a culture of the strain, a concentrate of the culture, and a dried product of the culture.

According to one embodiment of the present disclosure, the microbial insecticide formulation may have insecticidal activity against thrips.

Yet another aspect of the present disclosure provides an insecticidal method comprising a step of treating soil or a plant with the microbial insecticide formulation.

### Advantageous Effects

The Beauveria bassiana KNU-101 strain of the present disclosure is entomopathogenic, exhibits an excellent insecticidal effect on controlling thrips, which are soil pests that harm crops, and also has excellent sporulation ability. Thus, the Beauveria bassiana KNU-101 strain may be effectively used as an environmentally friendly means for controlling pests.

### BRIEF DESCRIPTION OF DRAWINGS

FIG. 1 shows the morphological characteristic (A) and microscopic photograph (B) of a Beauveria bassiana KNU-101 strain of the present disclosure after pure isolation. FIG. 2 shows a phylogenetic systematic diagram prepared using a gene encoding an internal transcribed spacer (ITS) region for the Beauveria bassiana KNU-101 strain of the present disclosure.

FIG. 3 shows the thrips killed by the Beauveria bassiana KNU-101 strain of the present disclosure, and the arrows in FIG. 3 indicate the thrips killed by the Beauveria bassiana KNU-101 strain.

FIG. 4 is a graph showing the comparison between the entomopathogenicity of the Beauveria bassiana KNU-101 strain of the present disclosure and those of other strains against thrips.

FIG. 5 is a graph showing spore production after 7 days of culture of the Beauveria bassiana KNU-101 strain of the present disclosure.

FIG. 6 is a graph showing the control value of the Beauveria bassiana KNU-101 strain of the present disclosure against thrips in a field experiment conducted using pepper.

### BEST MODE

The present inventors have searched for entomopathogenic microorganisms for controlling pests that harm various commercially important agricultural crops, and as a result, have found that the Beauveria bassiana KNU-101 strain has an excellent effect of killing thrips and excellent sporulation ability, and have prepared a microbial insecticide formulation for pest control containing the strain, thereby completing the present disclosure.

In one example of the present disclosure, thrips that are soil pests were treated with the Beauveria bassiana KNU-101 strain, and as a result, it was confirmed that the Beauveria bassiana KNU-101 strain showed a thrips mortality of 100%, indicating that the strain has excellent entomopathogenicity against thrips (see Example 2).

In another example of the present disclosure, it was confirmed that the sporulation ability of the Beauveria bassiana KNU-101 strain was at least 100 times to up to 10,000 times higher than those of other entomopathogenic fungi (see Example 3).

In still another example of the present disclosure, it was confirmed that the control value of the Beauveria bassiana KNU-101 strain was about 30% higher than those of other entomopathogenic fungi (see Example 4).

Hereinafter, the present disclosure will be described in detail.

One aspect of the present disclosure provides a Beauveria bassiana KNU-101 strain (accession number: KCTC 13660BP) having an insecticidal effect.

According to one embodiment of the present disclosure, the strain may comprise an ITS (Internal Transcribed Spacer Region) nucleotide sequence represented by SEQ ID NO: 1.

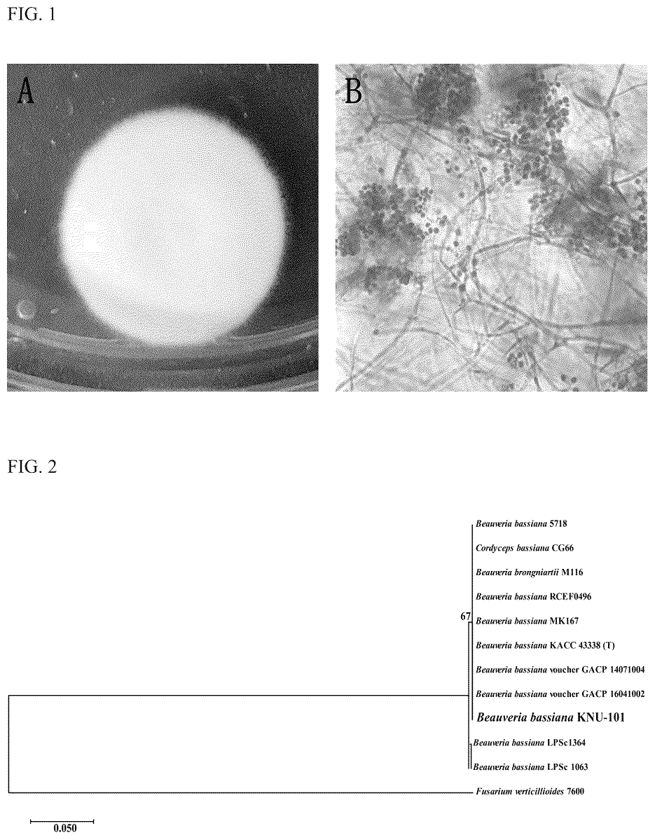

In the present disclosure, strains having an insecticidal effect against thrips were isolated from the thrips infected and killed by entomopathogenic fungi, and crops and soil inhabited by thrips, and the insects infected and killed by entomopathogenic fungi and the soil surrounding the insects were collected, and strains having an insecticidal effect against thrips were isolated therefrom. From these strains, a strain exhibiting an excellent insecticidal effect against thrips was selected (see FIG. 1). The selected strain was named ‘Beauveria bassiana KNU-101’ (see FIG. 2), and deposited with the Korean Collection for Type Cultures at the Korea Research Institute of Bioscience and Biotechnology, 181 Ipsin-gil, Jeongeup-si, Jeollabuk-do 56212, Republic of Korea, on Oct. 16, 2018 (accession number: KCTC 13660BP).

The Beauveria bassiana KNU-101 strain of the present disclosure may have optimal growth conditions at 30° C. and pH 7.0, and is an aerobic fungus that may be freeze-dried and stored. The strain may be stored by a conventional method. For example, the strain may be stored by mixing a culture of the fungus with a 40% glycerol aqueous solution containing 0.02% polysorbate 80 at a ratio of 1:1, followed by freezing at-80° C.

Another aspect of the present disclosure provides a method for mass production of a Beauveria bassiana KNU-101 strain (accession number: KCTC 13660BP), the method comprising a step of culturing the strain.

To a basal medium containing a mixture of water and bran for mass-producing the Beauveria bassiana KNU-101 strain of the present disclosure in an economical manner, one or two or more selected from the group consisting of, but not limited to, glycerin, glucose, yeast extract, and phosphate, may be additionally added. Factors that have the greatest influence on the growth of the fungus during culture on a solid medium are air permeability, temperature, and water content. When the temperature is 30° C., as the contact of the fungus with oxygen becomes smoother, the number of viable cells produced increases and the amount of cells produced also increases. The yeast extract added to the medium serves to provide trace elements and nitrogen sources and to maintain the insecticidal activity of the activated strain, the glycerin serves as an auxiliary to improve the physical properties and water content of the medium, and the phosphate serves as a pH buffer.

According to one embodiment of the present disclosure, the culturing may be performed at 28° C. to 30° C.

Still another aspect of the present disclosure provides a microbial insecticide formulation containing, as an active ingredient, at least one selected from the group consisting of a Beauveria bassiana KNU-101 strain (accession number: KCTC 13660BP), a culture of the strain, a concentrate of the culture, and a dried product of the culture.

The microbial insecticide formulation may be prepared by adding additives, such as a surfactant, an extender and a nutritional agent, to a Beauveria bassiana KNU-101 strain (accession number: KCTC 13660BP), a culture of the strain, a concentrate of the culture, or a dried product of the culture. At this time, as the surfactant, there may be used at least one selected from the group consisting of polycarboxylate, sodium lignosulfonate, calcium lignosulfonate, sodium dialkyl sulfosuccinate, sodium alkyl aryl sulfonate, polyoxyethylene alkyl phenyl ether, sodium tripolyphosphate, polyoxyethylene alkyl aryl phosphoric ester, polyoxyethylene alkyl aryl ether, a polyoxyethylene alkyl aryl polymer, polyoxyalkylene alkyl phenyl ether, polyoxyethylene nonyl phenyl ether, sodium sulfonate naphthalene formaldehyde, Triton 100 and Tween 80. As the extender and the nutritional agent, there may be used at least one selected from the group consisting of bean flour, rice, wheat, red clay, diatomaceous earth, dextrin, glucose, and starch. As a disintegrant, there may be used at least one selected from the group consisting of bentonite, talc, dialite, kaolin, and calcium carbonate.

The microbial insecticide formulation may be prepared in the form of, but not limited to, granules, wettable powders or encapsulated formulations for the purpose of stable formulation of the strain. The granules may be prepared to further contain at least one selected from the group consisting of a surfactant, an inert carrier, a preservative, a humectant, a feeding stimulant, an attractant, an encapsulating agent, a binder, an emulsifier, a dye, a UV protectant, a buffer, a flow agent, driftless dust, flow dust, and spreading oil.

According to one embodiment of the present disclosure, the microbial insecticide formulation may have insecticidal activity against thrips.

The Beauveria bassiana KNU-101 strain (accession number: KCTC 13660BP) of the present disclosure shows an excellent control rate against thrips, which is about 30% higher than those of strains of the same species, and thus may be effectively used to control thrips.

Yet another aspect of the present disclosure provides an insecticidal method comprising a step of treating soil or a plant with the microbial insecticide formulation.

The insecticidal method of the present disclosure may be performed by treating soil or a plant with a microbial insecticide formulation containing, as an active ingredient, at least one selected from the group consisting of a Beauveria bassiana KNU-101 strain (accession number: KCTC 13660BP), a culture of the strain, a concentrate of the culture, and a dried product of the culture, according to a conventional method known in the art.

### Mode for Invention

Hereinafter, preferred examples will be presented to aid in the understanding of the present disclosure. However, the following examples are provided only for easier understanding of the present disclosure, and the scope of the present disclosure is not limited by the following examples.

### EXAMPLES

### Example 1. Isolation and Identification of Beauveria bassiana KNU-101 Strain

In the present disclosure, in order to isolate fungi having insecticidal activity against soil pests, both a method of collecting the insects infected and killed by pathogenic fungi outdoors and a method of isolating fungi from the crop and soil samples inhabited by soil pests were performed.

Specifically, the infected and killed thrips are weighed and then diluted serially 10-fold in 0.85% saline solution. 0.1 ml of each dilution was taken and streaked on the Sabouraud dextrose agar (SDA) medium shown in Table 1 below. The medium components shown in Table 1 below are merely examples of components for isolating the Beauveria bassiana strain of the present disclosure, and may be changed without limitation.

TABLE 1

| Component | Amount (%) |
|---|---|
| Glucose | 4.0 |
| Yeast extract | 1.0 |
| Peptone | 1.0 |
| Polysorbate 80 (tween 80) | 0.01 |
| Ampicillin (100 mg/mL) | 0.01 |
| Kanamycin (100 mg/mL) | 0.01 |
| Agar powder | 1.5 |
| pH 6.9 ± 0.2 | |

In addition, in order to isolate fungi from soil, crop, feces and feed samples, 1.0 g of each collected sample was sequentially diluted in 9 ml of 0.85% saline solution, and 0.1 ml of each of the serial dilutions was taken, streaked on the medium having the composition shown in Table 1 above, and cultured at 30°° C. for 5 days. Then, only fungi morphologically similar to generally known Beauveria bassiana were selected, isolated, inoculated onto fresh SDA media, and cultured under the same conditions.

As a result, 13 entomopathogenic fungi having insecticidal activity against thrips were selected, and the fungi were identified as Beauveria bassiana. The present inventors named the fungus showing the best insecticidal activity among the above isolated entomopathogenic fungi as ‘Beauveria bassiana KNU-101’, and deposited Beauveria bassiana KNU-101 with the Korean Collection for Type Cultures at the Korea Research Institute of Bioscience and Biotechnology, 181 Ipsin-gil, Jeongeup-si, Jeollabuk-do 56212, Republic of Korea, on Oct. 16, 2018 (accession number: KCTC13660BP).

FIG. 1A shows the morphological characteristics of the Beauveria bassiana KNU-101 strain after pure isolation, FIG. 1B shows a microscopic photograph of the Beauveria bassiana KNU-101 strain after pure isolation, and FIG. 2 shows a phylogenetic systematic diagram prepared based on the results of sequencing the internal transcribed spacer (ITS) region. The phylogenetic systematic diagram of the strain was constructed by the maximum likelihood method using Molecular Evolutionary Genetics Analysis 7.0 version.

### Example 2. Verification of Pathogenicity of Beauveria bassiana KNU-101 Strain against Soil Pests

Among thrips as pests that become a problem in cultivation of cucumbers, tomatoes, lettuce and the like, Palm thrips (Parthenothrips dracaenae) were used as test insects. In addition, kidney beans were used as a host crop for adult insects. An experiment was conducted within 2 to 4 generations while zone breeding was performed.

In order to verify the insecticidal activity of the fungus Beauveria bassiana, isolated in the present disclosure, against thrips, about 10⁴ spores of the fungus were inoculated into Sabouraud dextrose liquid medium and pre-cultured, and about 10⁵ spores of the fungus were cultured on a solid medium made of bran for 5 days. After culture, as a result of observation with a hemocytometer, it was confirmed that about 3.1×10⁷ spores per gram were obtained. Thrips obtained from farms were treated with about 105 spores of the obtained spores and observed for 7 days. Each of an untreated group and treated groups consisted of about 30 thrips. Aspergillus flavus ATCC 9643 was used as a negative control, and Beauveria bassiana ATCC 74040 was used as a positive control.

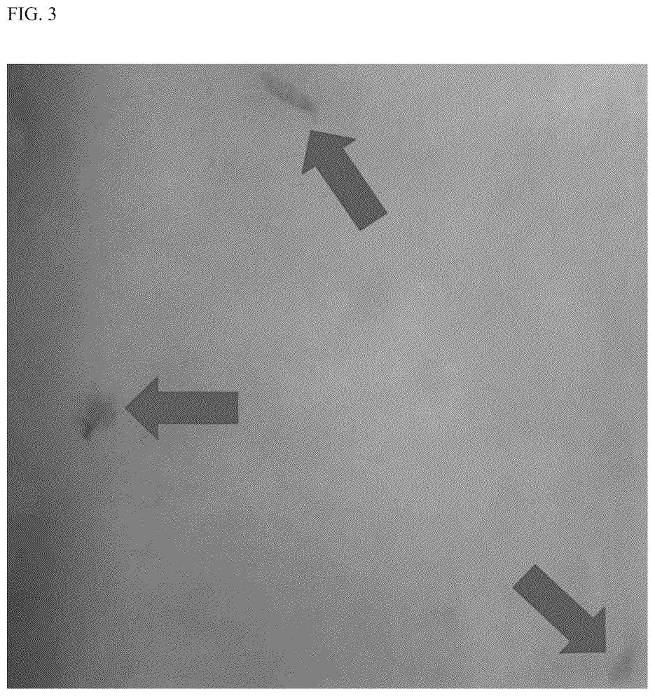

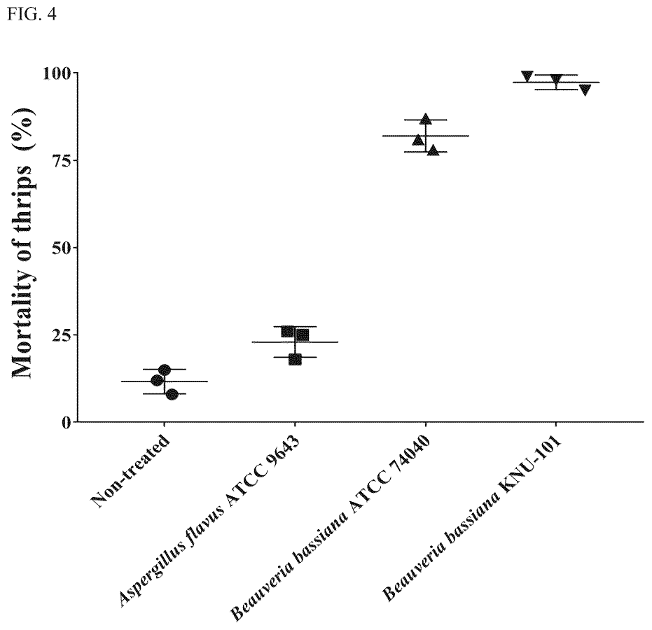

As a result, as shown in FIGS. 3 and 4, it was confirmed that the Beauveria bassiana KNU-101 strain showed a thrips mortality of 100%, indicating that the entomopathogenicity of the strain against thrips is better than those of other isolated fungi and the positive control.

Example 3. Comparative Verification of Sporulation Ability of Beauveria bassiana KNU-101

In order to mass-produce the Beauveria bassiana KNU-101 strain of the present disclosure in an economical manner, a basal medium containing a mixture of water and bran was prepared.

To the medium, one or two or more selected from the group consisting of glycerin, Bombycis corpus, yeast extract, and phosphate may be additionally added. Among the components added to the medium, the yeast extract serves to provide trace elements and nitrogen sources; the glycerin serves as an auxiliary to improve the physical properties and water content of the medium; the Bombycis corpus serves to maintain the insecticidal activity of the activated strain; and the phosphate serves as a pH buffer.

As test strains, Aspergillus flavus ATCC 9643 and Beauveria bassiana ATCC 74040 were used. Each of these strains and the Beauveria bassiana KNU-101 strain isolated in the present disclosure was inoculated onto a solid medium at a spore concentration of about 10⁴ spore/ml per gram of the medium and cultured for 7 days, and then the number of spores was counted. For these strains, three groups were set.

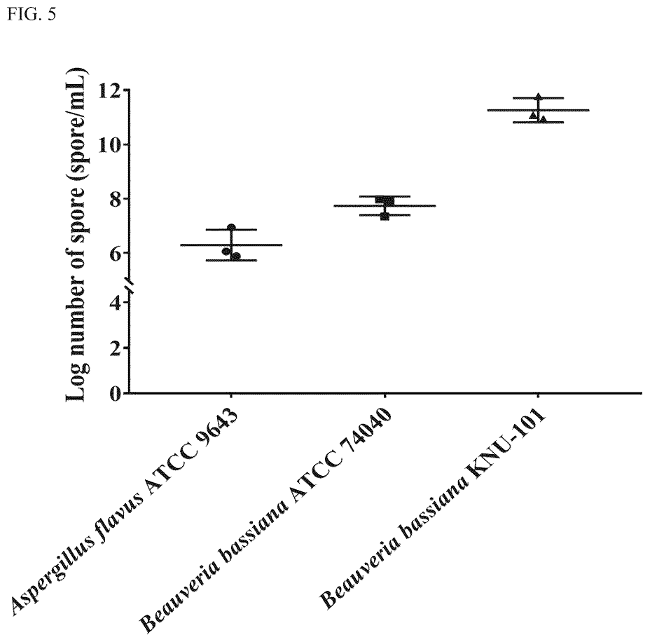

As a result, as shown in FIG. 5, when observed with a hemocytometer after 7 days of culture at 28 to 30° C., about 1.8×10¹¹ spores were found in gram of the medium on which the Beauveria bassiana KNU-101 of the present disclosure was cultured. In addition, it was confirmed that the sporulation ability of the Beauveria bassiana KNU-101 strain was at least 100 times to up to 10,000 times higher than that of the test strain Beauveria bassiana ATCC 74040.

### Example 4. Demonstration of Control Effect of Beauveria bassiana KNU-101

An empirical experiment was conducted to verify the control value of Beauveria bassiana KNU-101, isolated in the present disclosure, against soil pests in actual crops.

The experiment was conducted for a total of 27 days using pepper as a test crop and Frankliniella occidentalis as a test insect, and done in triplicate with a completely randomized design in a total of 45 m².

Treatment with the Beauveria bassiana KNU-101 strain of the present disclosure was performed as shown in Table 2 below, and Beauveria bassiana ATCC 74040 was used as a positive control. In a field experiment, pots planted with pepper seedlings were placed in test plots blocked from outside, and 300 Western flower thrips (Frankliniella occidentalis) bred indoors were inoculated into each test plot. The control value was determined by examining the density rate of Western flower thrips in the field 27 days after the start of the experiment.

TABLE 2

Efficacy test

| | Active | Dilution | Treatment |
|---|---|---|---|
| | ingredient | factor and | timing |
| Test agent | content (%) | amount used | and method |
| ATCC 74040 | 50% | 300 g/10a | Soil drench |
| granule | | | treatment after |
| formulation | | | planting |
| KNU-101 | 50% | 300 g/10a | Soil drench |
| granule | | | treatment after |
| formulation | | | planting |
| Untreated | — | — | — |

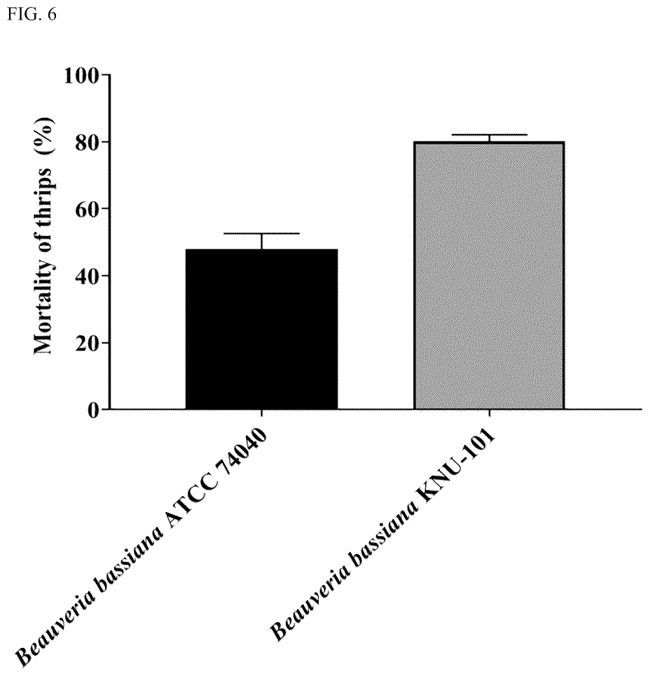

As a result, as shown in FIG. 6, it was confirmed that the KNU-101 formulation of the present disclosure showed a control value (mortality of thrips) of about 80% and had higher insecticidal activity than the positive control strain even in the actual field experiment.

The above description of the present disclosure is for illustrative purposes, and those skilled in the art to which the present disclosure pertains will understand that the present disclosure may be easily embodied in other specific forms without departing from the technical spirit or essential characteristics of the present disclosure. Therefore, the embodiments described above should be considered to be illustrative in all respects, not restrictive.

What is claimed is:

1. An insecticidal method comprising a step of treating soil or a plant with a microbial insecticide formulation,
wherein said formulation comprises one or more selected from the group consisting of a Beauveria bassiana KNU-101 strain deposited under accession number: KCTC 13660BP, a culture of the strain, a concentrate of the culture, and a dried product of the culture, as an ingredient.

2. The insecticidal method of claim 1, wherein said formulation has insecticidal activity against thrips.

3. The insecticidal method of claim 1, wherein said Beauveria bassiana KNU-101 strain is cultured in a medium comprising a mixture of water and bran, and one or more selected from the group consisting of glycerin, Bombycis corpus, yeast extract, and phosphate.

4. The insecticidal method of claim 1, wherein said formulation is a granule formation.
